(19) United States
(12) Patent Application Publication
(10) Pub. No.: US 2025/0100811 A1
(43) Pub. Date: Mar. 27, 2025

(54) Apparatus and a method for processing beverage containers

(71) Applicant: KRONES AG, Neutraubling (DE)

(72) Inventors: Hans-Juergen FLEISCHMANN, Steinberg am See (DE); Thomas GERSTENBERG, Regensburg (DE); Thomas HOELLRIEGL, Teublitz (DE); Andreas KURSAWE, Straubing (DE)

(21) Appl. No.: 18/893,027

(22) Filed: Sep. 23, 2024

(30) Foreign Application Priority Data
Sep. 21, 2023 (DE) 10 2023 125 643.2

(51) Int. Cl. B65G 43/00; B65G 13/11

(52) U.S. Cl. CPC B65G 43/00; B65G 13/11; B65G 2201/0244; B65G 2203/0266

(57) ABSTRACT

An apparatus for processing containers having a transport device which transports the containers to be processed along a predefined transport path has at least one processing device which is configured to process a container in a predefined manner, wherein the apparatus has a movement device, having at least one first guide cam and at least one guide roller which is configured to roll relative to the guide cam. The apparatus also has a detection device which is configured to detect at least one measurement value which is characteristic of a running property of the guide roller relative to the guide cam, and an evaluation device which is configured to evaluate this detected measurement value taking into account at least one further operating value characteristic of the operation of the apparatus.

### BACKGROUND OF THE INVENTION

The present invention relates to an apparatus and to a method for processing beverage containers. Various apparatuses and methods of this type have long been known in the prior art. For example, forming devices such as stretch blow molding machines are known which forms plastic material preforms into plastic material bottles. In addition, also inspection devices are known that inspect containers or labeling devices that apply a label to containers.

In these apparatuses, the containers to be processed are usually transported and a processing operation is carried out on these containers, such as a labeling process, a forming process, or the like. For this purpose, it is common for processing elements such as a blow nozzle to be placed on the plastics material preforms in order to then expand them.

In the prior art, different approaches to realizing these movements are known. Thus, it would be possible to assign individual drives, such as electric motors, to each processing element. However, a long-standing, proven approach is to provide guide cams on which guide rollers roll, and the position of the guide cam determines the position of the processing element. This technology is proven and has long been standard in such machines.

However, such cam systems are subject to wear and tear, which over time can lead to movements no longer being carried out precisely.

From DE 10 2017 120 295 A1 a container processing system and a method for detecting running properties of a roller on a guide cam of such a container processing system are known. This document describes that individual movements are recorded by sensor devices so that information can be determined about wear of the lifting cam, hereinafter referred to as the guide cam, or of the rollers, hereinafter referred to as the guide rollers.

The disclosure content of DE 10 2017 120 2951 is also incorporated in its entirety into the subject of the present patent application by reference.

From the applicant's internal prior art, it is known to provide such machines with vibration sensors on guide courses such as the main cam, a locking cam, and a stretching rod cam. If certain limit values are exceeded, depending on the limit exceeded, either warning messages are displayed (for example on a display device) or production is stopped and the machine is shut down or an emergency stop of the machine is initiated.

However, there are various possible causes of these increased vibration levels. If a certain limit is exceeded, an emergency stop is intended to prevent machine damage.

One problem here is that when traveling through the guide cams, a plurality of guide rollers, for example of blowing stations, are engaged at the same time. Due to this simultaneous engagement of a several guide rollers in the cam, an increased vibration value cannot always be reliably attributed to a specific forming station or processing station in general.

Since the error cannot be reliably assigned to a specific processing station, the search for the cause of the error is more complex because several processing stations have to be checked.

Nor can any conclusions be drawn about the state of individual guide cams.

In the prior art, a response therefore occurs when a determined limit value is exceeded. Possible influences on the vibration values, such as the customer object produced, the machine speed, the running time of the machine, or environmental conditions are not taken into account.

In addition, a history, for example a trend, is not taken into account in order to be able to predict the remaining running time or even the time until a failure occurs.

The present invention is therefore based on the object of facilitating or simplifying an assessment or prognosis of occurring errors.

### SUMMARY OF THE INVENTION

An apparatus according to the invention for processing containers has a transport device which transports the containers to be processed along a predefined transport path and at least one processing device which is suitable and intended to process at least one container and in particular at least one container transported by the transport device in a predefined manner, wherein the apparatus has a movement device (which is preferably suitable and intended to bring about a relative movement, and in particular between the container to be processed and the processing device and/or at least one element of this processing device in a direction of movement).

It should be noted that in some types of machines the transport device can preferably also be a processing device. For example, the forming stations act on the one hand as processing devices which form plastics material preforms into plastics material containers, but on the other hand they also act as transport devices because they transport the plastics material preforms along a predefined transport path (in particular during processing).

This movement device has at least one first guide cam and at least one guide roller which is suitable and intended to roll relative to the guide cam (preferably, this guide roller rolls relative to at least one rolling surface of the guide cam). Furthermore, the apparatus has at least one detection device which is suitable and intended to detect at least one measurement value which is characteristic of a running property of the guide roller relative to the guide cam.

A running property of the guide roller in relation to the guide cam refers in particular to any characteristic of this rolling, such as possible imbalances during rolling, vibrations caused by a damaged surface of the guide roller, vibrations caused by a damaged surface of the guide cam and the like.

According to the invention, the apparatus has an evaluation device which is suitable and intended to evaluate this recorded measurement value taking into account at least one further operating value characteristic of the operation of the apparatus and/or a container processing system, wherein the apparatus is a component of this processing system.

It is therefore proposed that the measurement values are not only recorded and stored as such and that information is derived directly therefrom, but that an evaluation is also carried out taking into account the aforementioned (operating) value characteristic of the operation of the apparatus. This characteristic (operating) value can, for example, be a production speed, as shown in more detail below, but also environmental values such as a temperature and the like.

In addition, this characteristic value can also be a value which is characteristic of the operation of the apparatus itself, such as a stretch rod position of a specific forming station (in particular a forming station to which a monitored guide roller is assigned or the like). In addition, the operating value can also be an operating value that is characteristic of a further apparatus (of the same container processing system). The operating value can also be a value that is characteristic of the container processed by the apparatus.

In a preferred embodiment, at least one roller is pressed and/or pushed against a guide cam with a predefined force (this can be the case, for example, with a locking and blow nozzle cam or, for example, with a labeling cam). Preferably, the roller is actively pressed against the cam (for example using a spring).

In a further embodiment, at least two rollers are positively guided in a cam and in particular a grooved cam (in the applicant's machines, this can be a main cam or a cam for moving pivot arms in a distribution delay starwheel).

In this case, a guiding and/or pushing and/or contact pressure occurs, in particular due to a sequence of movements.

The invention is therefore based on the idea that not only the movement of the guide rollers themselves is monitored, but this movement is also monitored in the context of other variables. This allows more precise statements to be made as to whether a specific signal from the detection device is due to a fault in a specific guide roller or has other causes that are caused by the operation of the apparatus or a higher-level container processing device.

Preferably, the evaluation device is suitable and intended to output a result value, in particular a result value from which a user can deduce how to proceed. For example, a result value may indicate that a guide cam of a particular forming station will soon fail. A result value can also indicate that a guide roller which is assigned to a specific holding device for holding containers will soon fail. The result value can also be fed into a system control, which then initiates corresponding controls of the container handling system, such as slower operation or a machine stop.

Particularly preferably, at least one holding device is provided, and a plurality of holding devices for holding individual containers is preferably provided. Preferably, the transport device has a plurality of such holding devices.

Preferably, the transport device has a movable and in particular rotatable carrier on which these holding devices are arranged.

In a preferred embodiment, each holding device is assigned a processing device. Conversely, each processing device is preferably assigned at least one and preferably exactly one holding device. In a further preferred embodiment, each processing device and/or each holding device is assigned at least one guide roller. However, it would also be possible for several guide rollers to be assigned to a specific processing device (for example to carry out different movements such as a closing movement of blow molds or a stretching rod movement).

The said cam roller is preferably connected either to the processing device or to the holding device associated with this processing device, so that the processing device or the holding device follows a movement of the cam roller (in a direction deviating from the transport direction of the containers). However, it would also be possible for both the holding devices and also the processing devices to be coupled with a cam roller.

Therefore, the guide cam causes, for example, a movement of the cam roller in a direction perpendicular to the transport path (of the containers), and the processing device coupled to the guide roller or the holding device for the containers follows this movement.

In a preferred embodiment, the direction of movement is perpendicular to the transport path of the containers. Preferably, the guide cam is stationary.

In a further advantageous embodiment, the apparatus has a second guide cam which controls or causes at least one further movement of the processing device or of a component of this processing device or of another processing device. Preferably, this second guide cam also has at least one detection device which is suitable and intended to detect at least one second measurement value which is characteristic of a running property of the roller relative to the guide cam.

Preferably, in this case, the evaluation device is suitable and intended to take into account the measurement values of both detection devices, wherein in this case the measurement value of the second detection device is the further (operating) value characteristic for the operation of the apparatus.

For example, in the case of a blow molding machine, an opening mechanism that opens and closes the blow molds can be driven by a guide cam and, on the other hand, also a movement of a stretching rod or also a blow nozzle, which is placed on the plastics material preforms to be expanded, can be moved by a (further) guide cam.

The present invention is described below with reference to a forming device for forming plastics material preforms into plastics material containers and also with reference to a labeling machine. However, it is pointed out that the invention is also applicable to other types of machines, such as filling machines, sterilization machines, or inspection machines.

In the case of a machine, detection apparatuses are already known from the applicant's internal prior art (in which a special cutout in the guide cam is provided, as well as a sensor). These are used in conjunction with a guide cam, in particular to allow monitoring of individual rollers.

Attempts have already been made to use this system in a first step in the main cam (a forming device). In addition, however, the possibility of using this system to detect a defect in a bearing of a (specific) guide roller is also to be created.

In a second step, an attempt is made to use this system for a blow nozzle cam and further cams, such as for a distribution delay starwheel.

Preferably, this described system is adapted, designed and integrated accordingly for all cam applications.

Preferably, the measurement values of these recording devices, for example vibration sensors, are transferred to a higher-level data acquisition system, in particular together with other machine parameters such as the machine speed or the machine performance, and preferably processed and stored there.

The at least one operating value can be a machine parameter or process parameter (e.g., a parameter of the furnace or the blow molding machine) which is selected from a group of machine parameters which includes a machine speed, a machine performance, a machine state (e.g., a year of manufacture and/or information on a retrofit and/or a machine state check), a machine type (e.g., information on a machine generation), a (current) time indication, a container size or preform size characteristic of a container that is to be or has been processed, and the like, as well as combinations thereof.

The container size can be a size characteristic of a container type (finished container or preform) and/or a container type and/or a container material and/or a composition of the container material (e.g., recycled material) and/or a (geometric) dimension of the container.

The at least one operating value can (additionally or alternatively) be an environmental variable which is characteristic of an environment in which the apparatus is set up. The environmental variable can be characteristic of an air humidity, a climatic condition or climate, an indoor and/or outdoor temperature (of a hall in which the apparatus is set up), an air pressure, a (geometric) variable characteristic of the positioning of the apparatus, such as a variable characteristic of a subsurface (slope, condition) of the machine hall, and the like, as well as combinations thereof.

It is possible for the measurement values of these vibration sensors to be preprocessed, for example in a control.

Preferably, the apparatus is designed in such a way that data processing is already carried out in a machine control or that parts thereof or only individual results are forwarded to a data acquisition system.

It is also possible for the complete processing to take place in a higher-level system (e.g., a production line, on the Internet, or in a cloud), which preferably processes and analyses all data from a plurality of machines in the line or production accordingly.

With the aforementioned detection device, for example a built-in vibration sensor in conjunction with a special cutout (the guide cam), increased vibrations, damage to the raceway of a roller, and damage to a bearing can be detected and preferably also assigned to a specific processing station, for example a forming station.

Troubleshooting is made much easier by the assignment to a specific forming station. Depending on the embodiment, wear on a specific guide roller can also be detected.

In a preferred embodiment, the apparatus has an assignment device which assigns a measurement value an operating value characteristic of the operation of the apparatus and in particular an operating value which is characteristic of a period or point in time at which the measurement value was recorded and/or at which the said measurement value occurred.

This assignment creates the possibility of seeing certain measurement values in a machine context, for example in connection with a current operating speed, but also in connection with environmental parameters. In this way, a much more accurate picture of the occurring measurement values can be drawn.

In a further preferred embodiment, the apparatus has a timer device which is suitable and intended to assign a measurement of a measurement value and/or the measurement value itself a time period and/or a point in time in which or at which this measurement value was measured.

In a further advantageous embodiment, the apparatus has a storage device which is suitable and intended for storing a plurality of measurement values, wherein these measurement values can preferably be stored with an assignment to operating values. In this way, a large plurality of data can be recorded, wherein the contexts in each case of the measurement values, such as operating states (and preferably also the times at which they occurred), are also stored.

In addition, further measurement values, for example of the same processing station, can also be assigned to individual measurement values. For example, a value of a stretch rod position or an (current) opening or closing state of a forming station can be assigned to a specific measurement value of a vibration sensor.

In a further advantageous embodiment, the apparatus has a plurality of processing devices and in particular a plurality of processing devices of the same type which are suitable and intended to process containers, and in particular containers transported by the transport device, in a predefined manner.

Preferably, at least one cam roller is assigned to each of these processing devices. For example, these processing devices can be forming stations that expand plastics material preforms into plastics material containers. Preferably, these processing devices are arranged on a common carrier, and in particular on a common movable and in particular rotatable carrier.

Preferably, these forming stations each have blow molds and/or blow mold carriers which can be pivoted to open and close the blow molds. This pivoting process is preferably effected by a guide cam and a guide roller rolling against this guide cam.

In addition, the forming stations can each have locking mechanisms which lock the side parts of the blow molds together when the blow molds are closed.

Preferably, these forming stations in each case have rod-like bodies, in particular so-called stretching rods, which can be inserted into the interior of the plastics material preforms in order to stretch them in their longitudinal direction. The movement of these stretching rods can also be effected by a guide cam and at least one cam roller assigned to the corresponding stretching rod.

Preferably, the forming stations each have application devices which can be placed on the plastics material preforms in order to apply a fluid medium and in particular pressurized air to them. This movement of the application devices can also be effected by a guide cam and a guide roller assigned to this guide cam.

Particularly preferably, these processing devices are arranged on a common, in particular movable and in particular rotatable, carrier, wherein the holding devices for holding the containers to be processed are preferably also arranged on this carrier. Preferably, each processing device is assigned exactly one holding device. This holding device can be designed as a gripping clamp, but it would also be conceivable for other elements, such as blow molds, to be used as holding devices.

In a preferred embodiment, each of the holding devices for holding the plastics material containers (or plastics material preforms) or generally each of the containers is assigned a guide roller which carries out at least one movement perpendicular to the transport path.

The processing devices and/or the holding devices are preferably arranged, as mentioned, on a common movable and in particular rotatable carrier.

Preferably, recorded and/or stored measurement values can also be used for a monitoring system and/or for outputting messages to a display device, for example a mobile phone, tablet, or similar. With these messages, certain groups of people, such as operating personnel, can carry out maintenance or the like or be informed about it.

The current measurement values of a vibration measurement, for example, can be put in relation to other values that influence the vibration values, such as the customer object produced, the plastics material preforms used or other container parameters. In addition, machine parameters, heating parameters, production parameters, and if necessary the running time of the machine in question or environmental conditions can also be used or set, allowing an evaluation to be carried out with dynamic and possibly also station-specific (limit) values.

In a further preferred embodiment, a history of the machine and/or apparatus can also be taken into account. In this way, an emerging trend can be identified at an early stage and, based on this, a forecast can be made of the remaining running time until a critical machine state occurs (predictive maintenance).

In a preferred embodiment, an algorithm is provided which can be trained based on the data of several machines or apparatuses in order to continuously learn during operation. In particular, artificial intelligence (AI) is used.

Preferably, all available data from an as largest possible number of containers as possible and a wide variety of production data and/or environmental data and/or measurement values (in particular of the measurement values characteristic of the physical properties of the support rings) are brought together and/or assigned to one another, and a model is preferably formed therefrom.

Various options can be considered for the modeling. For example, classic correlation analyses or dimensional analyses can be carried out. Relationships can also be modeled using mathematical fitting functions.

A model can also be created, in particular with the help of an expert. Also conceivable are various AI methods, such as a neural network, reinforcement learning, or physically based AI, which generate a model.

Preferably, the determination of the at least one result value, preferably of operating parameters and/or environmental parameters, is carried out with a, preferably trainable, machine learning model, which is preferably based on an (artificial) neural network and in particular on at least one, and in particular exactly one, (artificial) neural network.

The data that flow into the model (as input variables) can be generated during standard production or in special production runs in which specific parameters are specifically varied.

It is also conceivable to combine both methods and to generate a basic set of data for a basic model in a “teaching run” and then gradually feed data into the system during ongoing production and, if necessary, refine the model.

The neural network is preferably designed as a deep neural network (DNN), in which the parameterizable processing chain has a plurality of processing layers, and/or a so-called convolutional neural network (CNN) and/or a recurrent neural network (RNN).

Preferably, the data (to be processed or preferably to be evaluated by the evaluation device), in particular the measurement value recorded by the detection device (preferably the measurement values recorded by the detection devices), such as the data of a vibration sensor (or data derived therefrom) and/or the data of the processing device and/or the at least one operating value (preferably a plurality of operating values) are fed to a model or the (artificial) neural network as input variables. The model or the artificial neural network preferably maps the input variables, dependent on a parameterizable processing chain, to output variables, wherein the result value is preferably selected as the output variable and a plurality of result values is preferably selected as output variables.

Preferably, the evaluation device, preferably processor-based, evaluates the at least one measurement value or the plurality of measurement values and the at least one operating value or the plurality of operating values by the model.

It is also conceivable that the model is fed not only with the input variables related to exactly one apparatus, but with the input variables related to at least two and preferably a plurality of apparatuses. Thus, for each apparatus a data set comprising at least one measurement value (detected by a detection device) and at least one operating value (preferably a plurality of operating values) could be supplied as input variables.

The data to be fed into the model as input variables can be retrieved and/or transmitted (at least in part and preferably all) from an external storage device. The external storage device can be a storage device that is external to the apparatus and is arranged, in particular, at a distance from the operating site of the apparatus. The external storage device can therefore advantageously be used to collect the data of a plurality of apparatuses (in particular independently of the operator of the apparatus) and evaluate them (for example by the external server).

Preferably, a plurality of input variables is fed to the model, all of which are and/or can be assigned to a predefined and/or predefinable period of time (in which they were collected or determined). Preferably, this plurality of input variables (of the same survey period) comprises at least 10, preferably at least 50, preferably at least 100 different input variables.

The plurality of apparatuses are preferably apparatuses for processing containers with the same (basic) processing objective, for example the formation of plastics material preforms into plastics material containers. It is also conceivable that several or all of the plurality of apparatuses are apparatuses of identical design. It is also conceivable that several or all of the plurality of apparatuses are structurally similar apparatuses or apparatuses of different generations. It is also conceivable that the plurality of apparatuses are apparatuses from the same manufacturer.

Preferably, several or all of the plurality of apparatuses are not arranged on the same premises and/or in the same machine hall.

Preferably, the plurality of apparatuses comprises at least two, preferably at least 5, preferably at least 10 and particularly preferably at least 100 apparatuses that are different from one another (pairwise).

Preferably, at least one result value is characteristic of an operating state and/or error state of the apparatus and/or a component of this apparatus and/or a component connected to the apparatus (such as a further processing apparatus located upstream or downstream in the transport direction of the containers).

Preferably, the model and/or the evaluation apparatus is suitable and intended to recognize—by carrying out pattern recognition from the plurality of input variables—patterns which lead to an operating state of the apparatus that deviates from an intended operating state and/or require or cause and/or bring about such an operating state.

An operating state deviating from an intended operating state is understood to mean, in particular, an operating state of the apparatus in which an error state and/or a critical operating state in which an error state will occur with a high (perhaps predefined) probability within a predefined and/or predefinable period of time is present. An operating state that deviates from an intended operating state can also be understood as an operating state that leads to and/or causes unintended and/or above-average wear and/or consumption of resources (such as energy consumption) that exceeds a predefined and/or predefinable level.

It is conceivable that—for example by an operator of the apparatus—it can be specified and/or predefined which operating states are to be assigned to the category of intended operating states and which operating states are to be assigned to the category of unintended operating states. For example, if an operating state occurs which the operator considers to be unintended, the operator could mark this operating state as unintended. This can be done in real time (e.g., by monitoring the apparatus) but also retrospectively (by marking historical data). Such a label can be stored with a time stamp together with the measurement values recorded in the same period and further data (such as operating values), and can be assigned thereto.

Preferably, in a pattern recognition phase, in which the evaluation apparatus obtains patterns, the evaluation apparatus carries out a pattern recognition with a first predefined data set of input variables as well as variable(s) assigned thereto characteristic of an operating state of the apparatus and/or in each case associated time stamps.

Preferably, depending on the patterns obtained, the evaluation apparatus carries out a classification, which is preferably carried out by assigning the input variables (assigned to a predefined period of time) to one or more classes. The classes are preferably characteristic of at least one operating state of the apparatus or at least one operating state that deviates from the intended operating state.

Preferably (during operation of the apparatus) upon detection of at least one pattern obtained by pattern recognition in the supplied input variables which leads to a predefined and/or predefinable probability of an operating state deviating from an intended operating state, a warning variable and/or notification variable is determined and/or provided for output (to the operator of the apparatus) and/or is output (by a display device).

It is conceivable that the evaluation apparatus, even after an initial pattern obtaining phase, continuously and/or at regular or predefined and/or predefinable time intervals analyzes the data (input variables) fed thereto (or to the model) for further and/or new patterns (to be obtained) or evaluates them for updating and/or improving the patterns. Preferably, the continuous pattern recognition or pattern obtaining takes place simultaneously with the identification of patterns currently occurring in the input variables. Preferably, the evaluation apparatus is suitable and intended, preferably after confirmation by an operator, to take new and/or modified patterns (obtained by pattern recognition) into account when evaluating the input variables supplied in the future and/or to no longer take previous patterns into account.

It is also conceivable that the essential input variable(s) leading to the recognized pattern are determined as (further) output variable(s) and are provided (to the operator of the apparatus) for output and/or are output (by a display device). In this way, they could be visually highlighted in a particular way.

Preferably, the system now adjusts the performance of the system parts and in particular the physical properties of the cam rollers or the guide cams to the target performance, in particular to the target properties of these cam rollers or guide cams, in particular on the basis of the existing model, during the production run (or during short breaks).

For this purpose, the processing device, in particular the heating device and/or the forming device and/or the labeling device, preferably attempts to adjust the controllable production data in such a way that the desired performance and/or is achieved. In particular, the operation of the container handling system can also be adapted to the actual state of guide rollers.

When it comes to production data, a distinction is preferably made between three different types of data and/or parameters, namely, on the one hand, data or parameters that can be directly influenced (e.g., machine speed or filling pressure or heating power), parameters that can be indirectly influenced (wall thickness distribution, preform temperature at the furnace outlet or filling level or the properties of the manufactured or labeled containers) and values that cannot be influenced (air humidity, IR absorption behavior, or hall temperature).

In order to achieve the desired target performance and/or target states, in particular with regard to the state of the cam roller and/or the guide cam, different cascaded models can be used. For example, it is conceivable that there is a model that adjusts an indirectly controllable variable (e.g., wall thickness distribution, or characteristic properties of the support ring) by adjusting directly controllable values (e.g., heating disc, heating devices or stretching speed or blowing pressures) in such a way that the design of the support ring stored in the main model is generated for the current hall temperature in order to achieve the target performance.

A further point that can preferably be included in the model is secondary conditions such as energy requirements or filling pressure or a force with which a blow nozzle is placed on the plastics material preforms to be expanded. For example, an attempt can be made to get as close as possible to the target performance with minimal energy consumption.

It is also conceivable that the model will be gradually refined by continually collecting performance data and comparing them with the model forecast data.

The present invention will now be explained using a specific example, namely a blow molding machine. Such blow molding machines have a rotatable transport carrier on which a plurality of forming stations is arranged, which form plastics material preforms into plastics material containers. Furthermore, these machines preferably also have stretching units which stretch the plastics material preforms in their longitudinal direction. Preferably, these machines also have process controls that regulate the forming processes in particular individually for each forming station.

In addition, as mentioned above, these machines preferably have heating devices which heat the plastics material preforms.

Preferably, a movement of the stretch rod device and/or the movement of a blow nozzle device and/or an opening and/or closing movement of a blow mold is effected by guide cams and guide rollers rolling on these guide cams.

During the process development or validation of the properties of the guide rollers and/or guide cams, it is preferable to remove several rollers and then subject them to quality tests. Optimization loops are preferably run iteratively until the recognition quality is found to be good.

If it turns out that the best result was already found at the beginning of this optimization loop and no further improvement could be found afterwards despite prolonged efforts, these initial results would also be accepted as a compromise.

In the following, offline measurements are understood to mean measurements on guide cams and/or guide rollers that are carried out outside of a manufacturing process and which ultimately are intended to prove whether a guide roller and/or a guide cam meets the required quality standards. These measurements include, for example, force measurements, vibration measurements, speed measurements and the like outside of a production facility.

Online measurements are understood to mean measurements that can be carried out during production and in particular without removing containers and/or plastics material preforms (such as in particular vibration measurements during running operation, force measurements during running operation, and the like).

If these measurements correlate sufficiently well with the actual quality requirements, offline measurements can be reduced or eliminated completely.

However, as mentioned above, offline measurements and online measurements that were carried out with respect to the same cam roller are preferably assigned to one another.

In a further preferred method, both offline measurements, i.e., measurements outside of the working operation, and online measurements, which are carried out in the production operation, are taken. Preferably, identification information can be specified or output both for these offline measurements and for online measurements.

The identification information or the reject ID makes it possible to exactly match the online control target with the “best offline measurement data.” Currently, the target values of the online measurement data are recorded by the measuring system during the learning phase (DoE (statistical design of experiments)) and are then passed on to the control system.

These are preferably not identical to the values that were achieved in the validation process, but “only” those online measurement values that result from repeating the best setting parameters (best set point settings) from the validation process in the teach-in phase (DoE).

Since the disturbing influences to be regulated can lead to a deterioration in the container quality even with identical setting parameters, there is a risk that the best possible container quality will not be stored as the control target during the learning phase, but rather a less favorable wall thickness profile. The control target would therefore be worse than desired by a customer.

However, if the corresponding identification information is available for the test rolls rejected in the validation process, not only the complete setting parameters of the stretch blow molding machine and/or the heating device (best target value settings), but also the associated online measurement data for these rolls can be transferred from the database alone, in conjunction with the associated time stamp data, back to the measuring unit and into the control system as a perfect control target.

Furthermore, it is possible for the data or measurement values obtained to be used for comparison and/or analysis purposes, in particular in order to be able to draw conclusions about different machine sizes, machine designs or even other machines, for example with regard to spare parts service life and to obtain production forecasts, and thus to be able to further increase benefits to the customer.

In a further advantageous embodiment, the apparatus has a comparison device which compares measurement values, for example vibration values, with at least one target value and preferably outputs a comparison value characteristic of this comparison. Preferably, this is used to output a comparison between actual values, for example of a vibration measurement, and target values. Preferably, this comparison value is a difference or a quotient or the like. Particularly preferably, this comparison value is stored.

In a further advantageous embodiment, the apparatus has a selection device that is suitable and intended for selecting a target value from a plurality of target values. This makes it possible, for example, to preselect a target value, for example depending on a container to be processed, a machine speed, or the like. For example, a plurality of target values for vibration measurements can be stored in a storage device, each of which is assigned to different operating speeds.

In a further advantageous embodiment, the apparatus has at least one further sensor device which is suitable and intended to record at least one value characteristic of influencing factors. Preferably, this value is selected from a group of values which include temperature values, pressure values, air humidity values, values which are characteristic of the containers to be processed (such as the material of the container and/or plastics material preform or a wall thickness or a weight), values which are characteristic, for example, of a label adhesive and the like.

Particularly preferably, the evaluation device is suitable and intended to also take these values into account.

In a further advantageous embodiment, the detection device has an evaluation module which is suitable and intended to evaluate measurement values—in particular taking into account an output of a rotary encoder—in order to assign the measurement values to the individual guide rollers on a guide cam or also guide rollers on different guide cams. Preferably, this rotary encoder is assigned to a rotatable carrier on which the individual processing devices are arranged.

In a further advantageous embodiment, the assignment can be carried out taking into account predefined data. The assignment can be carried out taking into account a specific group of data or also taking into account different types of data.

Preferably, the data are selected from a group of data which contains an angle of rotation, in particular of a transport carrier (or a processing device) or a position of a transport device, the position of a processing device and/or a time information, or a signal of a rotary encoder.

In this design, it is in particular determined at which point in time a certain measurement value was evaluated. In addition, a rotary encoder can be used to determine which processing station (and thus also which cam roller(s)) was at the position of the sensor device at that time.

In a further advantageous embodiment, the apparatus is selected from a group of apparatuses which includes forming devices for forming plastics material preforms into plastics material containers, filling devices for filling containers, sterilizing devices for sterilizing containers, heating devices for heating plastics material preforms, printing devices for printing containers, labeling devices for labeling containers, closing devices for closing containers with container closures, and cleaning devices for cleaning containers.

In a further advantageous embodiment, the detection device has at least one sensor device which detects at least one measurement value which is characteristic of at least one movement variable of the guide roller or the processing devices or the holding device. Particularly preferably, this sensor device is selected from a group of sensor devices which includes speed sensors, force sensors, acceleration sensors, vibration sensors and the like.

For example, the occurrence of a certain vibration on a guide cam can indicate wear on a guide roller rolling on this guide cam (but possibly also wear on the guide cam itself).

The present invention is further directed to a container processing system having at least one apparatus of the type named above. According to the invention, the container processing system has a further apparatus for processing containers, which is preferably suitable and intended to process the containers in a different way than the said apparatus, and a (further) detection device is preferably also provided, which is suitable and intended to detect at least one measurement value characteristic of this further apparatus, wherein the evaluation device is preferably suitable and intended to also take this further measurement value into account in the evaluation.

This embodiment takes into account that values from other machines in a container handling system can often influence the corresponding apparatus and the values recorded by the recording device. For example, the values of a heating device for plastics material preforms can have an impact on the actual forming of the containers.

Preferably, the measurement values mentioned can be stored together with further machine parameters. Particularly preferably, a higher-level data acquisition system is provided which processes and stores these measurement values.

Particularly preferably, a processing on the container processing system or the machine is possible. Particularly preferably, only individual data points are transferred.

In a further advantageous embodiment, a higher-level system is provided in which the data can be processed. It is also possible for data or measurement values from several machines or apparatuses to be processed and analyzed.

In a further modified embodiment, the said detection device is arranged on the guide cam in such a way that the detection device can selectively detect the running property for each individual roller, wherein these individual rollers roll on the guide cam.

In a further advantageous embodiment, the guide cam has a first guide cam region and a second guide cam region. Advantageously, these first and second guide cam regions are (mechanically) decoupled from one another.

Particularly preferably, a sensor of the detection device is arranged in one of the two guide cam regions and in particular in the second (or first) guide cam region.

In a further preferred embodiment, the detection device has a detection path which (mechanically) couples a first guide cam region and a second guide cam region to one another, wherein a sensor device of the detection device for detecting vibrations is preferably arranged in the second guide cam region and is provided in particular for detecting those vibrations which are injected into the detection path by a guide roller.

Particularly preferably, the first guide cam region and the second guide cam region are decoupled from one another by a recess and/or a gap. Particularly preferably, the first guide cam region and the second guide cam region are connected to one another only in the region of a detection path.

In a further advantageous embodiment, the detected path is designed such that the second guide cam region of a predefined detection direction vibrates relative to the guide cam region when a roll rolls on the detection path.

Particularly preferably, the detection path is insensitive to vibrations injected in a remaining rolling path of the guide cam. In this way, the detection device can specifically assign occurring vibrations to a specific guide roller. This is preferably also done by evaluating signals from a rotary encoder.

In a further advantageous embodiment, this path has a length which corresponds at least to an outer circumference of a guide roller which rolls on the path. In this way, a precise assignment to a specific guide cam and thus preferably also to a specific processing device is possible.

In a further advantageous embodiment, the predefined detection device is arranged perpendicular to a plane in which the guide rollers roll along the detection path.

In a further advantageous embodiment, the extension of the second guide cam region in the direction of the length of the detection path is greater than the length of the detection path.

In a further advantageous embodiment, the detection device is designed as a sensor which has an acceleration sensor which is in particular suitable and intended for detecting acceleration amplitudes.

In a preferred method, the evaluation device is an external evaluation device, in particular a cloud-based and/or an external server. An external server is to be understood in particular to mean an external server, in particular a backend server, in relation to the apparatus. The external server is, for example, a backend of a manufacturer of the apparatus or of a service provider, which is set up to evaluate and/or manage and/or store measurement values and/or data derived therefrom and/or error states of the apparatus and/or operating states of the apparatuses. The functions of the backend or the external server can be carried out in (external) server farms. The (external) server can be a distributed system. The external server and/or backend can be cloud-based.

The present invention is further directed to a method for processing containers, wherein a transport device transports the containers to be processed along a predefined transport path and at least one processing device processes at least one container and preferably a container transported by the transport device in a predefined manner, wherein the apparatus has a movement device which preferably causes a relative movement between the container to be processed and the processing device in a direction of movement.

Furthermore, this movement device has at least one first guide cam and a guide roller which rolls in relation to the first guide cam. Furthermore, the apparatus has a detection device which detects at least one measurement value which is characteristic of a running property of the guide roller relative to the guide cam.

According to the invention, the apparatus has an evaluation device which evaluates this recorded measurement value taking into account at least one further value characteristic of the operation of the apparatus.

Particularly preferably, a further parameter or value characteristic of the processing is recorded, and preferably this further parameter is taken into account in the evaluation.

This further characteristic value can be, for example, a temperature, a pressure, a humidity, or the like.

Particularly preferably, the value characteristic of the operation of the apparatus and/or the further characteristic value or parameter is taken into account in the determination of target values.

### BRIEF DESCRIPTION OF THE DRAWINGS

Further advantages and embodiments result from the accompanying drawings.

In the drawings:

FIG. 1 is a highly simplified plan view of a container processing system according to an embodiment;

FIG. 2 is a side view of a very schematically illustrated holder for containers on a guide cam with a detection device of the apparatus according to the embodiment;

FIG. 3 is a plan view of the very schematically illustrated holder for containers on the guide cam of the apparatus according to the embodiment;

FIG. 4 is a simplified partial plan view of the guide cam with the detection device of the apparatus according to the embodiment;

FIG. 5 is a further simplified partial plan view of the guide cam with the detection device of the apparatus according to the embodiment;

FIG. 6 is a representation of the sensitivity of a detection path of the detection device according to the embodiment for different frequencies;

FIG. 7 is a schematic block diagram of the detection device according to the embodiment;

FIG. 8-10 each show a frequency spectrum of detection results at different points of the detection path of the detection device according to the embodiment;

FIG. 11 is a further simplified partial plan view of the guide cam with the detection device of the container processing system according to the embodiment;

FIG. 12, 13 each show an evaluation diagram of the detection device of the container processing system according to the embodiment;

FIG. 14 is a side view of a guide cam;

FIG. 15 shows a plan view of the cam shown in FIG. 14; and

FIG. 16 shows a detail view of the guide cam of FIG. 15.

### DETAILED DESCRIPTION OF THE INVENTION

In the figures, identical or functionally identical elements are given the same reference numerals unless otherwise indicated.

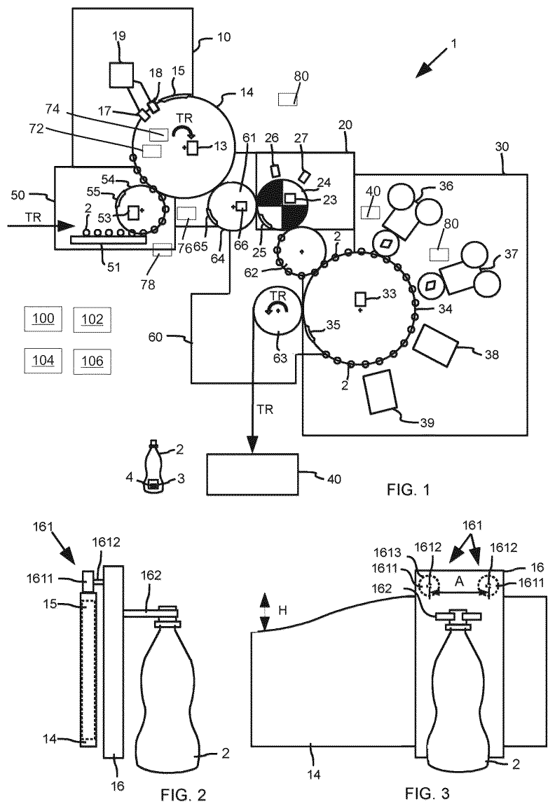

FIG. 1 shows a container processing system 1 for processing containers 2, which can be bottles, cans, boxes, containers, barrels, etc., and which can preferably be equipped with a very schematically shown label 3 and/or any printing 4 as identification.

For this purpose, the container processing system 1 has, as an example, first to fourth apparatuses 10, 20, 30, 40 for processing containers (hereinafter referred to as apparatuses for short), which are preferably coupled and/or synchronized with one another. However, the container processing system 1 can also have only one or two or three of the apparatuses 10, 20, 30, 40. There can also be more apparatuses 10, 20, 30, 40. Therefore, any number of apparatuses 10, 20, 30, 40 is possible. In FIG. 1, for the sake of simplicity, not all containers 2 are shown or provided with a reference sign.

These individual apparatuses 10, 20, 30, 40 preferably each have transport devices (not designated by their own reference signs) which transport the containers during their processing. These transport devices preferably each have transport starwheels or transport wheels, which are provided with holding devices for holding the containers.

At least one of the apparatuses 10, 20, 30, 40 is an apparatus according to the invention. Preferably, several of the apparatuses 10, 20, 30, 40 are apparatuses according to the invention and particularly preferably all of the apparatuses 10, 20, 30, 40 are apparatuses according to the invention.

A container 2 can, for example, be a container that has a maximum capacity of approximately 0.33 liters or 0.5 liters or 1.5 liters, etc. Other capacities are also conceivable. The shape of the container 2 can be freely selected. In addition, the material of the container 2 can be freely selected, such as glass, plastics material, in particular PET, aluminum, etc.

In the inlet of the first apparatus 10, a transport device 50 is provided which, with the aid of a transport rail 51 and a guide cam 54, hereinafter also referred to as a lifting cam, feeds containers 2 to the first apparatus 10 in a row or as a container stream. The guide cam 54 forms a rotary conveyor device, which preferably also has a rotary encoder 53. The guide cam 54 is furthermore preferably provided with a detection device 55.

The reference sign 100 refers to an evaluation device which, as described in more detail below, is suitable and intended to evaluate measurement values of the detection device(s), wherein in the context of this evaluation, further values characteristic of the operation of one or more apparatuses for processing containers are also taken into account, such as values from individual or several sensors of a labeling device, as described in more detail below.

The reference sign 102 designates an assignment device which assigns an operating value to the measurement value(s) which is characteristic of the operation of the corresponding apparatus or of the entire container processing system. This can be an operating value that comes from the same apparatus on which the measurement value is also measured, or from another of the apparatuses. In addition, the operating value can also be an environmental value, such as an ambient temperature, or an operating value that is characteristic of the entire container handling system, such as a production speed. This operating value is preferred.

In particular, this operating value can also be stored with a temporal assignment, i.e., it is preferably recorded at the time or in the period at which the measurement value was also recorded.

Preferably, a storage device 104 is also provided in which a plurality of measurement values can be stored, in particular with the operating values assigned to these measurement values and preferably also with a time for the occurrence of these values.

The reference sign 106 designates a comparison device which is suitable and intended to compare recorded measurement values with target values. Preferably, these target values are selected by a selection device from a plurality of target values. This selection is particularly preferably made using artificial intelligence.

The first apparatus 10 is preferably designed as a rotary machine with a guide cam 14, in which a detection device 15 is preferably provided.

In addition, the second apparatus 20 in the example of FIG. 1 has a rotary encoder 23 and a guide cam 24 with a detection device 25. In addition, in the example the third container processing machine 20 preferably has a rotary encoder 33 and a guide cam 34 with a detection device 35.

The transport direction TR of the containers 2 is in each case indicated by arrows and also results from the row of containers 2 along the apparatuses 10, 20, 30, 40.

Between the third and fourth apparatuses 30, 40, the containers 2 are transported by a transport device 60 with the aid of transport modules 61, 62, 63. The transport module 61 has a guide cam 64, in which a detection device 65 and a rotary encoder 66 are provided.

These transport modules are preferably designed as transport starwheels, which have a rotatable carrier on which a plurality of holding devices for holding the plastics material preforms or plastics material containers are arranged. In a preferred embodiment, at least one of these transport modules is designed as a distribution delay starwheel, which is suitable and intended to change a distribution or a distance of successively transported plastics material preforms or plastics material containers.

Otherwise, the transport devices 50, 60 in the example in FIG. 1 have multifunctional starwheels or carousels for transporting the containers 2. It is also possible that at least one of the apparatuses 10, 20, 30, 40 is alternatively designed as a multifunctional starwheel or carousel or has such.

In the example of FIG. 1, the apparatuses 10, 20, 30, 40 and the transport devices 50, 60 form a block which is very compact. The fourth device 40 can also be arranged near or adjacent to the block consisting of the apparatuses 10, 20, 30 and the transport devices 50, 60, even if this is not shown in.

However, the container processing system 1 and the apparatuses 10, 20, 30, 40 are not limited thereto and can also be arranged in series in a separate design.

In the example of FIG. 1, the first apparatus 10 is a filling machine in which the containers 2 are moved to very schematically illustrated filling valves 17, 18, with which at least one product from at least one filling tank is filled into the containers 2 fed by the transport device 50. Preferably, the filling valves move with the containers. The product can be, for example, a foodstuff, in particular a beverage, etc., or a cleaning agent or cosmetic product or a powder or bulk product, in particular beads, etc., or any other product that can be filled into container 2.

A sensor 72 for measuring the fill level of the product in the filling tank 19 can also be provided on the filling tank 19. The reference sign 74 designates a further sensor which is suitable and intended for detecting a valve position of a filling valve.

Preferably, the values or data output by these sensor devices 72, 74 can be used by the evaluation device as further values characteristic of the operation of the apparatus. Preferably, however, this filling tank can also be arranged on a rotatable carrier and thus rotate therewith.

In the example of FIG. 1, the second container processing machine 20 is a closing machine, which is connected downstream of the filling machine or first container processing machine 10. In the closing machine as second container processing machine 20, the containers 2 are provided with a closure cap with the aid of, for example, at least one screw drive device 26, 27, and are thereby closed. For simplicity, further components of the second container processing machine 20 are not shown and described.

The reference sign 76 designates a further sensor which detects at least one value which is characteristic of a closing process of the containers. This can be, for example, a rotational position of a closure relative to a container to be closed. Preferably, values output by this further sensor can also be used as further values by the evaluation device for evaluation.

In the example of FIG. 1, the third apparatus 30 is an equipping machine which equips the containers 2 filled by the filling machine or first apparatus 10, and then containers 2 closed with the second apparatus 20, with a marking by, for example, the label 3 and/or any desired printing 4. For this purpose, the equipping machine in the example of FIG. 1 has the guide cam 34. On the guide cam 34, the containers 2 are guided by holders past four different modules, which are in particular equipping modules 36, 37, 38, 39. For example, the equipping module 36 is a labeling and/or printing unit, the equipping module 37 is a labeling unit, and the equipping modules 38, 39 are designed as printing units. Accordingly, label 3 can be a pre-made label. The label can also be a self-adhesive label.

The reference sign 78 designates a sensor which is suitable and intended to detect at least one value characteristic of the equipping process, such as a temperature of a labeling glue or a rotational speed of a labeling cylinder. Preferably, values output by this further sensor can also be used as further values by the evaluation device for evaluation.

However, the label 3 can also be at least partially printed by a labeling and/or printing unit, which is illustrated by the printing 4. Alternatively, the labeling and/or printing unit can also print at least partially directly onto the label 3 or the container 2. In particular, the labeling and/or printing unit or units can be used with a printer, such as an inkjet or laser printer, to print an additional marking on the containers 2 to ensure traceability of the containers 2 or products.

Alternatively, however, the equipping modules 35 to 38 can each be designed identically, so that all containers 2 are provided with the same type of markings, in particular labels 3. In the case shown in FIG. 1, the containers 2 can in particular be provided with different types of labels, such as belly and/or chest labels and/or wrap-around labels from the roll or as a sheet and/or self-adhesive labels and/or cold-glue labels and/or hot-glue labels, etc. Any combination of equipping modules 35 to 38 and thus types of markings, in particular types of labels, are conceivable and feasible.

For the sake of simplicity, further components of the third apparatus 30, such as in particular print heads, sensors, drive devices for label rolls, etc. are not shown and described.

After the third apparatus 30, the equipping machine in the previously selected example, the containers 2 are fed to the fourth apparatus 40 by the transport device 60, as already mentioned above.

The fourth apparatus 40 is, for example, a packaging machine in which the containers 2 are packaged in predefined types and/or packaging sizes, for example as a bundle with two containers 2 or four containers 2, with or without a handle, etc., or in a box or carton, etc. A shrinking station can be provided for producing, for example, bundles or also for the cartons. The packaging machine can also be provided with a lifting cam or guide cam with a detection device, even if this is not shown in FIG. 1.

The apparatus 40 also preferably has one or more sensor devices which record values characteristic of the operation of this apparatus. This can, for example, be a value that is characteristic of the packaging process, such as the temperature of a shrink tunnel. Preferably, values output by this further sensor can also be used as further values by the evaluation device for evaluation.

The reference sign 80 roughly schematically designates a sensor device which is suitable and intended to record environmental values, such as an ambient temperature, an ambient pressure and the like. It would also be possible for each of the apparatuses 10, 20, 30, 40 to be assigned such a sensor device which records these environmental values (in particular locally related to this apparatus).

Preferably, values output by this sensor device can also be used as further values by the evaluation device for evaluation.

The previously described filling valves 17, 18, the drive devices 26, 27, the modules 36 to 39 and the retaining clamps described below are processing elements 17, 18, 26, 27, 36 to 39, 162 of the container processing system 1 or its corresponding apparatuses 10, 20, 30, 40.

Above, the invention has been explained with reference to a container processing system which has a filling device, a closing device, a labeling device and a packaging device as apparatuses for processing containers.

However, the invention is also applicable to other container processing systems, for example a system which has an apparatus for heating plastics material preforms, a forming device for forming plastics material preforms into plastics material containers, a sterilization device for sterilizing plastics material preforms and/or plastics material containers, and/or an inspection device for inspecting containers.

Sensors can be provided which detect other values, such as positions of stretch rods, blowing pressures, heating performances of heating elements of the heating device, and the like.

FIG. 2 shows, as an example, a highly simplified sectional view of the guide cam or lifting cam 14 with the detection device 15. The guide cams 24, 34, 54, 64 with the associated detection devices 25, 35, 55, 65 are preferably designed in the same way, so that the following description of the guide cam 14 and the detection device 15 also applies to the guide cams 24, 34, 54, 64 with the associated detection devices 25, 35, 55, 65.

A holder 16 is provided on the guide cam 14 for transporting a container 2 along the guide cam 14. The holder 16 has at least one (guide) roller 161, which rolls on the guide cam 14. More precisely, in the example of FIG. 2, a roller body 1611 of the at least one (guide) roller 161 rolls on top of the guide cam 14. For this purpose, the roller body 1611 is preferably rotatably attached to the holder 16 via an axle 1612. In addition, the holder 16 preferably has a holding device and in particular a holding clamp 162 for holding the container 2. The holder 16 can be preloaded downward on the guide cam 14, for example by compression springs (not shown) or otherwise, so that the at least one (guide) roller 161 runs securely on the guide cam 14.

As can also be seen from FIG. 3, in a plan view of another portion of the guide cam 14, a plurality of rollers 161 usually runs on the guide cam 14 simultaneously. The (guide) rollers 161 roll with the outer circumference 1613 of the roller body 1611

The (guide) cam 14 is preferably shaped such that the holder 16 is adjustable in height H. Alternatively, it is possible, for example, to adjust the height of a centering bell which serves to center the containers 2 on a turntable. Any other desired applications for the guide cam 14 are conceivable.

The (guide) rollers 161 each inject vibrations into the guide cam 14. The vibrations can be detected by the detection device 15 as structure-borne sound or acceleration. To ensure that the structure-borne sound is not perceptible only as broadband noise, the guide cam 14 and the detection device 15 are designed as described below. This makes it possible to use the detection device 15 to detect the running characteristics of an individual (guide) roller 161 on the guide cam 14.

The guide cam 14, like any other of the previously mentioned guide cams, can alternatively be designed in segment form. In this case, the rollers 161 do not run on a circle closed by the guide cam 14, but only on a predefined circle segment. The guide cam 14, like all the other aforementioned guide cams, can alternatively or additionally have at least partially a linear or oval portion, if this appears suitable for the particular application.

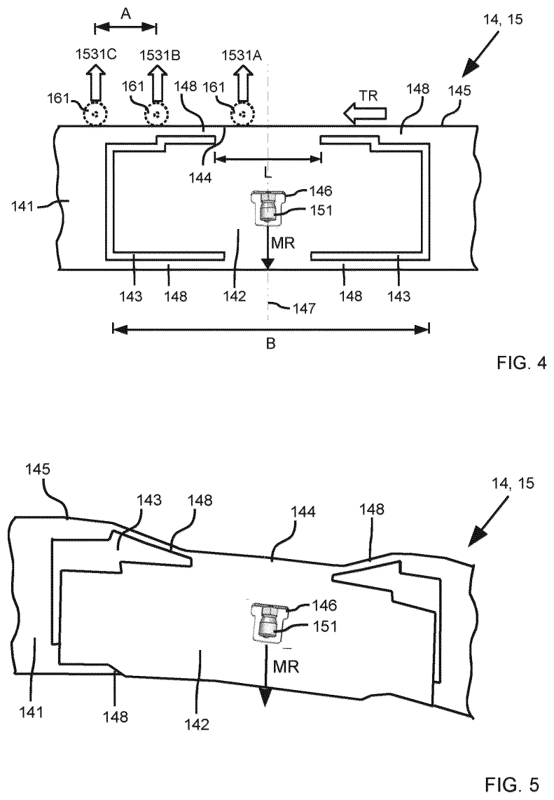

According to FIG. 4, the detection device 15 is partially integrated into the guide cam 14, which is specially designed for this purpose. The guide cam 14, which is only partially shown in FIG. 4, has a first guide cam region 141, which in the example is shown as a circle segment.

However, the guide cam 14 can form a full circle which includes the first guide cam region 141. In addition, the guide cam 14 has a second guide cam region 142, which in the example is made in one piece with the first guide cam region 141. The first and second guide cam regions 141, 142 are partially decoupled from one another by a first recess 143. The first and second guide cam regions 141, 142 are connected to one another only in the region of a detection path 144 and are thus coupled to one another.

The detection path 144 with a length L is provided along a rolling path 145 of the at least one roller 161 on the guide cam 14. In the example of FIG. 4, the rolling path 145 is arranged on top of the guide cam 14. A sensor 151 of the detection device 15 is arranged in a second recess 146 of the second guide cam region 142.

The first and second recesses 143, 146 are each arranged symmetrically to a line of symmetry 147 of the detection path 144 in the running direction of the roller 161 on the rolling path 145. The line of symmetry 147 is also the line of symmetry of the second guide cam region 142. Thus, the second recess 143 is arranged in the middle of the second guide cam region 142. The second guide cam region 142 has a maximum extension B in the running direction of the at least one roller 161.

The maximum extension B of the second guide cam region 142 in the direction of the length L of the detection path 144 is greater than the length L of the detection path 144. In addition, the recess 143 is designed in a stepped manner in the region perpendicular to the detection direction MR or parallel to the rolling path 145 and the detection path 144. In the example of FIG. 4, the second guide cam region 142 has two steps starting from the line of symmetry 147 up to a part of the recess 143 which is arranged approximately parallel to the line of symmetry 147. The second guide cam region 142 is wider in its middle at the symmetry line 147 than at its ends. The exact design of the guide cam region 142 can be freely selected within the framework of the following conditions.

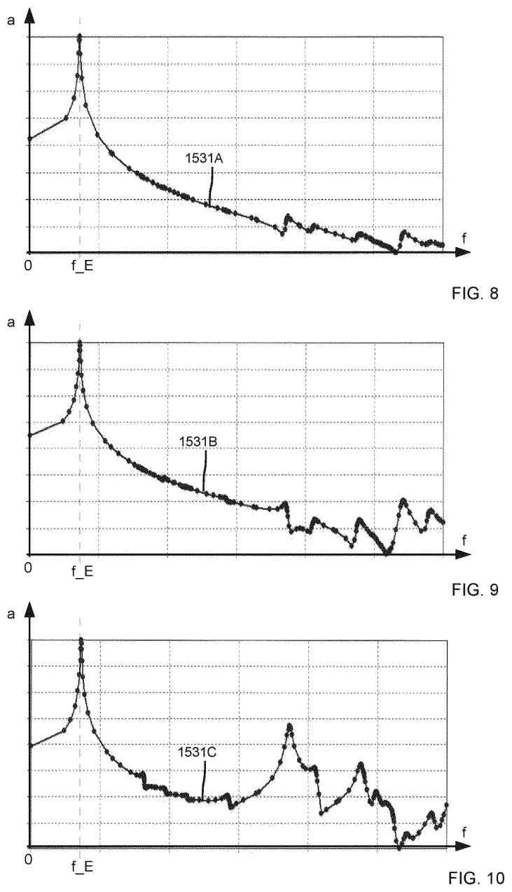

If the at least one roller 161 runs at a distance A from an optional next roller 161 along the rolling path 145 in the transport direction TR, the sensor 151 successively records different detection results 1531A, 1531B, 1531C, which are explained in more detail with reference to FIG. 8 to FIG. 10.

In order to evaluate the state, in particular the running properties, of an individual roller 161, the rolling path 145 is designed in its mechanical structure such that the sensor 151 in the recess 146 is very sensitive to structure-borne sound in the region of the detection path 144. In contrast, the sensor 151 in the recess 146 can hardly detect structure-borne noise outside the detection path 144, since excitations outside the detection path 144 are strongly suppressed or dampened.

The vibrational decoupling of the second guide cam region 142, including the sensor 151 arranged therein in the recess 146, from the first guide cam region 141 is achieved by a targeted design of the mass of the second guide cam region 142 and the rigidity of the connection of the first and second guide cam regions 141, 142 to one another.

As can be seen in a combined view of FIG. 4 and FIG. 5, the connection 148 of the second guide cam region 142 is selected such that a low rigidity results in a predefined detection direction MR in which the vibrations are detected. In the other two directions of the three-dimensional space, the connection 148 of the second guide cam region 142 is designed to be more rigid than in the detection direction MR. In comparison, the connection 148 of the second guide cam region 142 is very rigid in the other two directions of the three-dimensional space. Depending on the application, the material of the detection path 144 can also be softer or have a lower rigidity than the material of the remaining rolling path 145 outside the detection path 144. The design of the connection 148 and the recess 143 ensure that the detection path 144 and the rolling path 145 are decoupled from one another in terms of vibration.

According to FIG. 4 and FIG. 5, the acceleration values are recorded perpendicular to the rolling path 145, which is used for the rolling of the running surface of the roller 161, or of the outer circumference of the roller body 1611. The rolling path 145 lies in a plane that is approximately perpendicular to the predefined detection direction MR.

The combination of mass and rigidity of the second guide cam region 142 including the sensor 151 results in a natural frequency with which the second guide cam region 142 vibrates in the detection direction MR, as illustrated in FIG. 5.

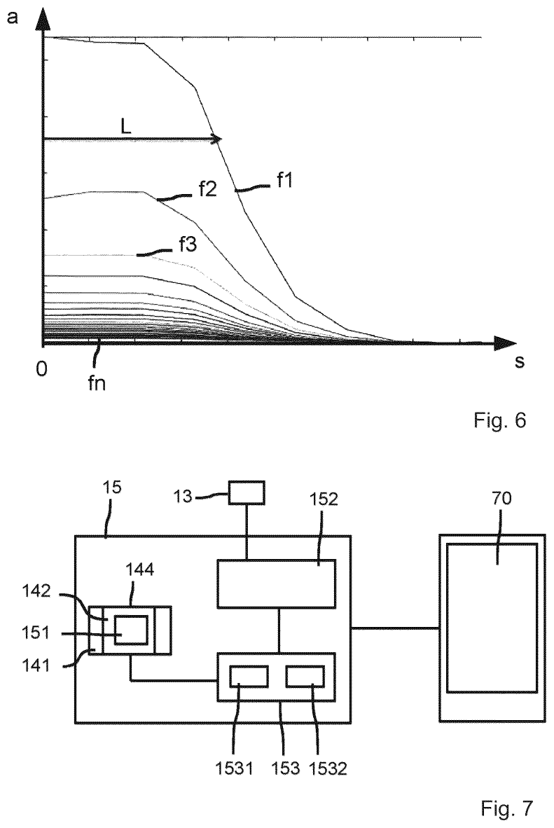

As shown in FIG. 6 for various vibration frequencies f1, f2, f3, . . . fn over the path s starting at 0 as the symmetry line 147, due to this arrangement, movements a, such as structure-borne sound or vibrations caused thereby with the frequencies f1, f2, f3, . . . fn, are very strongly suppressed, which movements both have their origin on a path s outside the detection path 144 (length L) and have a higher frequency f than the natural frequency of the guide cam region 142 including the sensor 151 and point in the direction of the detection direction MR.

On the other hand, movements a such as structure-borne sound or vibrations caused thereby with the frequencies f1, f2, f3, . . . fn, which have their origin in the length L of the detection path 144 and point in the detection direction MR, will result in a high amplitude of the acceleration a, as shown in FIG. 6.

The detection path 144 is designed to have a length between the recess 143 such that the length L of the detection path 144 corresponds minimally to exactly one deployment of one revolution of the roller body 1611 of a roller 161. The length L of the detection path 144 thus corresponds at least to the outer circumference of the roller body 1611 of a roller 161. The maximum length L of the detection path 144 depends on the distance A between the rollers 161, which is explained in more detail below.

As a result, the detection device 15 detects, as the detection results 1531A to 1531C, acceleration amplitudes having a higher frequency than the natural frequency of the second guide cam portion 142 including the sensor 151.

According to FIG. 7, the detection device 15 has, in addition to the sensor 151, an evaluation module 152 and a memory module 153. The sensor 151 is designed in particular as an acceleration sensor. The detection results 1531A to 1531C of the sensor 151 are stored as the actual value or detection result 1531 in the memory module 153. In addition, at least one target value 1532, which corresponds to a detection result of an ideal roller 161, is stored in the memory module 153. The target value 1532 is in particular a reference curve. The detection result 1531 or the detection results 1531A to 1531C and the target value 1532 are each assigned to an output of the rotary encoder 13.

Thus, the evaluation module 152 can evaluate the detection result 1531 or the detection results 1531A to 1531C and the target value 1532 in the correct position relative to one another on the guide cam 14. This allows the detection results 1531A to 1531C to be assigned to the individual rollers 161 on the guide cam 14.

The evaluation result and/or the detection result 1531 or the detection results 1531A to 1531C and the target value 1532 can be transmitted to an operating device 70. This makes it possible for an operator of the container processing system 1 to be informed on request or automatically about the running properties of at least one of the rollers 161. The state of wear of the corresponding roller 161 can be derived from the running characteristics by comparing it with the target value 1532. For example, a predefined tolerance band can be defined for a level of wear that is still tolerable. If the running characteristics of the roller 161 under consideration are outside the tolerance band, the roller 161 is assessed as worn out. Otherwise, the roller 161 under consideration can continue to be used. However, instead of a tolerance band, it is possible to specify only a limit beyond which the roller 161 is assessed as worn out.

FIG. 8 to FIG. 10 show examples of the outputs of the detection results 1531A to 1531C. Here, only the vibration or acceleration a is shown in relation to a frequency f. The detection result 1531A according to FIG. 8 corresponds to a detection of the vibrations or their frequencies f at the detection point in the measuring direction MR of the sensor 151, when a vibration with the acceleration a, always having the same amplitude but having variable frequency f, is coupled in by a roller 161 at a position which is offset by an angle of approximately 4° with respect to the line of symmetry 147, as shown in.

The detection result 1531B according to FIG. 4 FIG. 9 corresponds to a detection of the vibrations or their frequencies f for a roller 161 which, instead of the position of 4° according to FIG. 8, is arranged at a position which is offset by an angle of approximately 16° with respect to the line of symmetry 147, as shown in. The detection result 1531C according to FIG. 4 FIG. 10 corresponds to a detection of the vibrations or their frequencies f for a roller 161 which, instead of the position of 4° according to FIG. 8, is/are coupled in at a position which is offset by an angle of approximately 28° with respect to the line of symmetry 147, as shown in FIG. 4.

FIG. 8 to FIG. 10 thus each indicate a frequency spectrum for the different positions of the rollers 161 on the guide cam 14. From this, the natural frequency f_E of the mechanical structure of the second guide cam region 142 can be clearly read, namely as the peak of the detectable amplitudes of the acceleration a over the frequency f at the respective positions 4°, 16°, 28°.

In FIG. 8 to FIG. 10, the vertical scales of the representation are chosen differently. Effectively, the amplitudes of the detection result 1531A for the acceleration a are the largest and the amplitudes of the detection result 1531C for the acceleration a are the smallest, even if this is not apparent from the direct comparison of FIG. 8 to FIG. 10. This is consistent with the illustration in FIG. 6, according to which the amplitudes of the detected acceleration a decrease with increasing distance from the line of symmetry 147.

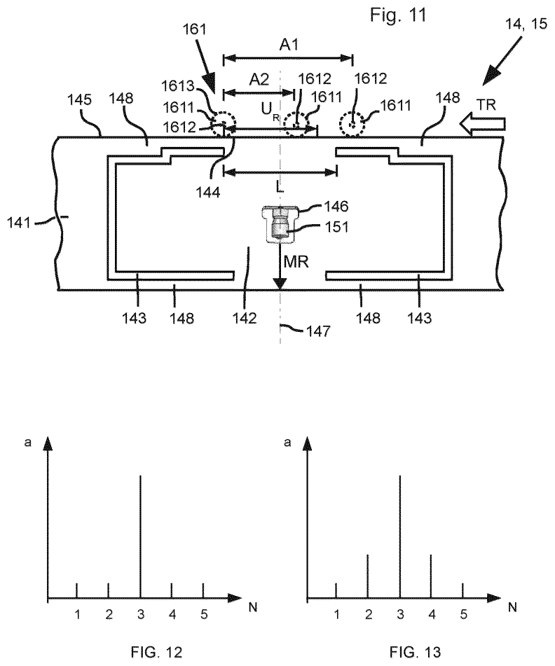

FIG. 11 illustrates two cases, namely a first case, in which there is a distance A1 between the axles 1612 of two consecutive rollers 161, and a second case, in which there is a distance A2 between the axles 1612 of two consecutive rollers 161. Here, the unwinding U of one R revolution of the roller body 1611, or the outer circumference 1613 of a roller 161, is smaller than the length L of the detection path 144.

The evaluation module 152 of the detection device 15 calculates the amplitude of the acceleration a or a detection signal as an effective vibration value for an evaluation angle interval of the machine 10 assigned to a roller 161, wherein one amplitude of the acceleration a is calculated for each roller 161. For each revolution of the machine 10 or for each revolution of a roller 161 around the machine 10, this amplitude of the acceleration a is recalculated and offset against previous detection values by an averaging process which can be carried out in particular using an averaging algorithm of the evaluation module 152.

The result of this evaluation is shown in FIG. 12 for five consecutive rollers 161 for the first case, in which there is the distance A1 between the axles 1612 of two consecutive rollers 161. In FIG. 13, the result of this evaluation for five consecutive rollers 161 is shown for the second case, in which there is the distance A2 between the axles 1612 of two consecutive rollers 161.

As can be seen from FIG. 12, in the first case of FIG. 11, a very sharp demarcation can be seen of the amplitude of the acceleration a of the third roller 161 on the detection path 144 from the amplitude of the acceleration a of the adjacent rollers 161, which are not arranged on the detection path 144. In this case, in the evaluation angle interval for the third roller 161 on the detection path 144, the vibrations that are measured are almost exclusively vibrations that originate from this roller 161 itself.

As can be seen from FIG. 13, in the second case of FIG. 11, a less sharp demarcation can be seen of the amplitude of the acceleration a of the third roller 161 on the detection path 144 from the amplitude of the acceleration a of the adjacent rollers 161. This is due to the fact that the second and/or fourth roller also roll over the detection path 144 at least partially at the same time as the third roller.

Thus, the acceleration amplitude of rollers 161 arranged adjacent to a worn-out roller 161 also increases. The reason for this is that the evaluation angle intervals for calculating an acceleration amplitude overlap. Thus, in the case illustrated in FIG. 13, the amplitude of the acceleration a for the second and fourth rollers 161 is also increased when the third roller 161 is worn out. However, the increase in the effective vibration value is still highest for the roller 161 being evaluated.

Thus, both for the first case of FIG. 11 and for the second case of FIG. 11, the selective evaluation of the running properties of the rollers 161 on the guide cam 14 is possible. In other words, the evaluation of the running properties of the rollers 161 on the guide cam 14 is also possible with the detection device 15 if the length L of the detection path 144 is smaller than the distance A( ) or A1( ) of the rollers 161 or if L<A or L<A1.

Thus, the detection device 15 detects and evaluates the vibrations generated by the rolling roller body 1611 of a single roller 161 in the second guide cam region 142 in the detection direction MR. In this case, the measured amplitudes of the acceleration a are assigned to a specific machine angle of the first container processing machine 10 or another rotary machine or rotary transport device such as the transport device 50 or the transport device 60 by including the output of the rotary encoder 13.

In this way, the selective evaluation of the running properties of at least one roller 161 of the rollers 161 can be carried out easily and safely in order to determine the state of wear of each individual roller 161 on the guide cam 14.

It is also possible to apply the principle described above to an arrangement in which the rollers 161 are not guided in a circular motion over a rolling path 145 but can be moved back and forth over a rolling path 145. Here, too, a detection path 144 can be provided which can detect the running characteristics of a single roller 161. Here, it is conceivable, if necessary, to use a recording device 15 to record only the running properties of at least one individual roller 161 out of all the rollers 161 running on the roller path 145. The running properties of the at least one individual roller 161 can then be used to infer the running properties of the other rollers 161. Even if this is less optimal in terms of recording the running properties for each individual roller 161, such an arrangement can still be sufficient if required, and in any case offers an improvement over the prior art.

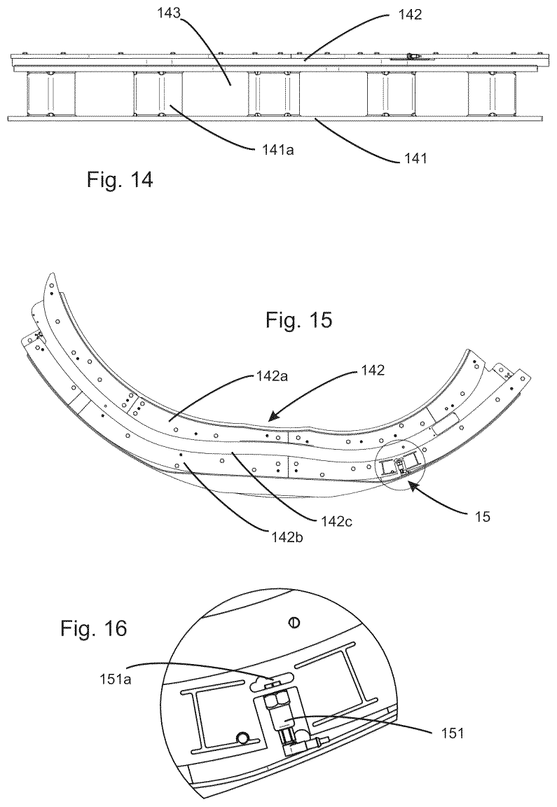

FIG. 14 shows another representation of a guide cam. This guide cam is intended in particular for the operation of a forming device for forming plastics material preforms into plastics material containers.

The guide cam has a first guide cam part 141 and a second guide cam part 142. These guide cam parts are spaced apart from one another by a gap 143 and connected to one another by connecting devices 141a.

A plurality of such connecting devices 141a are preferably provided at regular intervals from one another.

FIG. 15 shows a plan view of the guide cam shown in FIG. 14, more precisely of the second guide cam part 142.

It can be seen that this second guide cam part 142 has a first guide web 142a and a second guide web 142b, between which a gap 142c is arranged that preferably has a uniform width. Within this web, cam rollers (not shown) can roll, preferably both relative to the first guide web 142a and relative to the second guide web 142b.

The reference number 15 designates the detection device.

FIG. 16 shows an enlarged representation of the detection device. The sensor 151 is also shown again here.

All previously described embodiments of the container processing system 1 and of the method carried out by it for detecting running properties of a roller 161 on a guide cam 14, 24, 34, 54, 64 can be used individually or in all possible combinations. In particular, the features of the previously described exemplary embodiments can be combined as desired, or also omitted. In addition, the following modifications are particularly conceivable.

The parts shown in the figures are schematic diagrams and may differ in exact design from the shapes shown in the figures as long as their previously described functions are guaranteed.

At least one of the container processing machines 10, 20, 30, 40 can be a stretch blow molding machine for producing containers 2 from preforms. At least one of the container processing machines 10, 20, 30, 40 can be a heating device used in the stretch blow molding machine for heating the preforms. At least one of the container processing machines 10, 20, 30, 40 can be an inspection machine for inspecting the containers 2 for defects. At least one of the container processing machines 10, 20, 30, 40 can be a cleaning machine for cleaning the at least one container 2.

Of course, all other container processing machines designed for the processing of containers 2 are also possible as container processing machine(s) 10, 20, 30. The container processing system 1 can have container processing machines 10, 20, 30, 40, and/or further container processing machines as described above in any number, combination and design, if necessary with transport devices 50, 60 arranged in front of and/or between and/or after them in any number, combination, and design.

The applicant reserves the right to claim all features disclosed in the application documents as essential to the invention, provided that they are novel over the prior art individually or in combination. It is also pointed out that features which can be advantageous in themselves are also described in the individual figures. The person skilled in the art will immediately recognize that a particular feature described in a figure can be advantageous even without the adoption of further features from this figure. Furthermore, the person skilled in the art will recognize that advantages can also result from a combination of several features shown in individual or in different figures.

1. An apparatus for processing containers, having a transport device which transports the containers to be processed along a predefined transport path and having at least one processing device which is configured for processing a container in a predefined manner, wherein the apparatus has a movement device, and this movement device has at least one first guide cam and at least one guide roller which is configured to roll relative to the guide cam and wherein the apparatus further comprises a detection device which is configured to detect at least one measurement value which is characteristic of a running property of the guide roller relative to the guide cam,
wherein
the apparatus has an evaluation device which is configured to evaluate this detected measurement value taking into account at least one further operating value characteristic of the operation of the apparatus.

2. The apparatus according to claim 1,
wherein
the apparatus has an assignment device which is configured to assign to the measurement value an operating value characteristic of the operation of the apparatus.

3. The apparatus according to claim 1,
wherein
the apparatus has a storage device which is configured for storing a plurality of measurement values.

4. The apparatus according to claim 1,
wherein
the apparatus has a plurality of processing devices which are configured to process containers in a predefined manner, wherein each of these processing devices is assigned at least one cam roller.

5. The apparatus according to claim 1,
wherein
the apparatus has a comparison device which is configured to compare measured measurement values with at least one target value.

6. The apparatus according to claim 1,
wherein
the apparatus has a selection device which is configured to evaluate a target value from a plurality of target values.

7. The apparatus according to claim 1,
wherein
the apparatus has at least one further sensor device which is configured to record at least one value characteristic for a for influencing factors.

8. The apparatus according to claim 1,
wherein
the detection device also has an evaluation module which is configured to assign measurement values to the individual rollers on the guide cam.

9. The apparatus according to claim 8,
wherein
the assignment can be carried out taking into account specified data.

10. The apparatus according to claim 1,
wherein
the apparatus is selected from a group of apparatuses which includes forming devices configured for forming plastics material preforms into plastics material containers, filling devices configured for filling containers, sterilizing devices configured for sterilizing containers, heating devices configured for heating plastics material preforms, printing devices configured for printing containers, labeling devices configured for labeling containers, closing devices configured for closing containers with container closures, packing devices configured for packing containers, and cleaning devices configured for cleaning containers.

11. The apparatus according to claim 1,
wherein
the detection device has at least one sensor device which is configured to detect at least one measurement value which is characteristic of at least one movement variable of the guide roller or the processing device or the holding device.

12. A container processing system having at least one apparatus according to claim 1,
Wherein
the container processing system has a further apparatus configured for processing containers and a detection device is provided which is configured to detect at least one further measurement value characteristic of this further apparatus.

13. A method for processing containers, wherein a transport device transports the containers to be processed along a predefined transport path and at least one processing device processes at least one container in a predefined manner, wherein the apparatus has a movement device, and this movement device has at least one first guide cam, as well as at least one guide roller which is configured to roll relative to the guide cam and wherein the apparatus further has a detection device configured detect at least one measurement value which is characteristic of a running property of the guide roller relative to the guide cam,
wherein
the apparatus has an evaluation device which is configured to evaluate this recorded measurement value taking into account at least one further value characteristic of the operation of the apparatus.

14. The method according to claim 13,
wherein
at least one further parameter characteristic of the processing is recorded and taken into account in the evaluation.

15. The method according to claim 13,
wherein
the further characteristic parameter is taken into account in the determining of target values.
